(19) United States
(12) Patent Application Publication
(10) Pub. No.: US 2009/0124803 A1
(43) Pub. Date: May 14, 2009

(54) PROCESS FOR PREPARATION OF ROSUVASTATIN

(76) Inventor: Pandurang Balwant Deshpande, Mumbai (IN)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/816,144

(22) PCT Filed: Aug. 9, 2005
(86) PCT No.: PCT/IN2005/000266

§ 371 Date: Aug. 13, 2007

(30) Foreign Application Priority Data
Mar. 22, 2005 (IN) 325/MUM/2005

(51) Int. Cl. A61K 31/505; C07D 239/28

(52) U.S. Cl. 544/322; 514/275

(57) ABSTRACT

The invention relates to commercially viable process for the preparation of Rosuvastatin by an early introduction of the correct absolute stereochemistry at C-5 (S) of Rosuvastatin side chain followed by regioselective chain extension using novel side chain building blocks. It is yet another object of the invention is to provide novel intermediates that may be used for the preparation of Rosuvastatin. Formula (I).

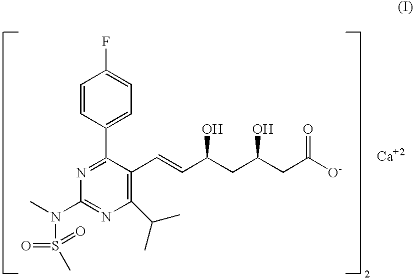

### FIELD OF INVENTION

The present invention relates to a process for the preparation of Rosuvastatin, a promising HMG-CoA reductase inhibitor, to process steps and novel intermediates.

### BACKGROUND OF THE INVENTION

HMG-CoA reductase inhibitors (also called β-hydroxy-β-methylglutaryl-co-enzyme-A reductase inhibitors and also called statins) are understood to be those active agents, which may be preferably used to lower the low-density lipoprotein (LDL) particle concentration in the blood stream of patients at risk for cardiovascular disease and thus used for the prevention or treatment of hypercholesterolemia, hyperlipoproteinemia and artheriosclerosis. A high risk level of LDL in the bloodstream has been linked to the formation of coronary lesions that obstruct the flow of blood and can rupture and promote thrombosis.

Rosuvastatin, which is an antihyperchlolesterolemic drug, is chemically (E)-7-[4-(4-flurophenyl)-6-isopropyl-2-[methyl(methylsulfonyl)amino]pyrimidin-5-yl](3R,5S)-3,5-dihydroxyhept-6-enoic acid calcium (2:1) salt having the structural formula I.

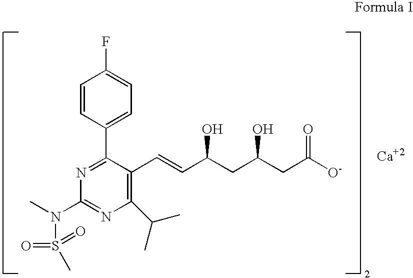

Rosuvastatin, its calcium salt (2:1) and its lactone form are disclosed and claimed in U.S. Pat. No. 5,260,440. The process of the '440 patent prepares rosuvastatin by reacting 4-(4-flurophenyl)-6-isopropyl-2-[methyl(methylsulfonyl)amino]pyrimidin-5- carbaldehyde with methyl (3R)-3-[(tert-butyldimethylsilyl)oxy]-5-oxo-6-triphenylphos phoranylidene hexanoate in acetonitrile under reflux. The silyl group is then cleaved with hydrogen fluoride, followed by regioselective reduction with sodium borohydride and diethylmethoxy borane to obtain a methyl ester of rosuvastatin.

The ester is then hydrolyzed with sodium hydroxide in ethanol at room temperature, followed by removal of ethanol and addition of ether, to obtain the sodium salt of rosuvastatin. The sodium salt is then converted to the calcium salt by adding calcium salt to the aqueous solution of sodium salt, resulting in precipitation of rosuvastatin calcium (2:1).

PCT publication WO 03097614 describes a modified procedure for the preparation of the starting material 4-(4-flurophenyl)-6-isopropyl-2-[methyl(methylsulfonyl) amino] pyrimidin-5-carbaldehyde and further conversion to rosuvastin by condensing with methyl (3R)-3-[(tert-butyldimethylsilyl)oxy]-5-oxo-6-triphenylphosphoranylidene hexanoate. The condensed product was deprotected using methanesulfonic acid and subsequently converted to rosuvastatin calcium (2:1) salt.

PCT publication WO 2004052867 describes a process to prepare rosuvastatin by condensing 1-cyano(2S)-2-[(tert-butyldimethylsilyl)oxy]-4-oxo-5-triphenylphosphoran-ylidene pentane with 4-(4-flurophenyl)-6-isopropyl-2-[methyl(methylsulfonyl)amino] pyrimidin-5-carbaldehyde and subsequent deprotection of silyl group, reduction and hydrolysis.

PCT publication WO 0049014 discloses a novel chemical process for the manufacture of tert-butyl (E)-(6-{2-[4-(4-flurophenyl)-6-isopropyl-2-[methyl(methylsulfonyl)amino] pyrimidin-5-yl]vinyl}-(4R,6S)-2,2-dimethyl[1,3]dioxan-4-yl)acetate which comprises reaction of diphenyl{4-(4-flurophenyl)-6-isopropyl-2[methyl(methylsulfonyl)amino] pyrimidin-5-yl-methyl}phosphineoxide with tert-butyl 2-[(4R,6S)-6-formyl-2,2-dimethyl-1,3-dioxan-4-yl]acetate and its further conversion to Rosuvastatin.

PCT publication WO 04014872 describes a process for the manufacture of Rosuvastatin calcium (2:1) salt which comprises mixing of a solution of calcium chloride with a solution of water soluble salt of (E) 7-[4-(4-flurophenyl)-6-isopropyl-2-[methyl (methylsulfonyl)amino]pyrimidin-5-yl](3R,5S)-3,5-dihydroxyhept-6-enoic acid.

The generation of phosphorane side requires eight synthetic steps and involves expensive reagents. The process is both uneconomical and time consuming, hence not appropriate for commercial scale operation.

It is therefore, desirable to provide an efficient and commercially viable method for the synthesis of Rosuvastatin.

### SUMMARY OF THE INVENTION

It is an objective of the present invention to provide a commercially viable process for the preparation of Rosuvastatin by an early introduction of the correct absolute stereochemistry at C-5 (S) of Rosuvastatin side chain followed by regioselective chain extension using novel side chain building blocks. It is yet another object of the invention is to provide novel intermediates that may be used for the preparation of rosuvastatin.

### DETAILED DESCRIPTION OF THE INVENTION

The present invention concerns a process for the preparation of rosuvastatin comprising, a) reacting a compound of formula (II)

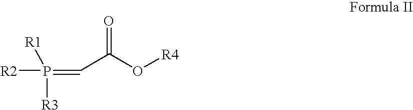

wherein, R1, R2, R3, are substituted or unsubstituted phenyl and R4 is an aliphatic residue selected from C1-C4 alkyl;
with a compound of formula R-CHO (III) wherein R represents the following structure (formula IV) to obtain a compound of formula V;

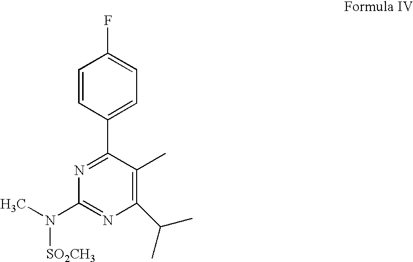

b). reducing a compound of formula (V) using diisobutylaluminium hydride (DIBAL) to obtain a compound of formula (VI);

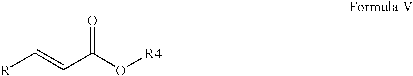

c). oxidising a compound of formula (VI) to obtain a compound of formula (VII)

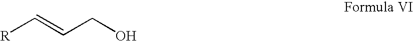

d). adding a compound of formula (VII)

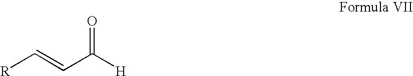

with a compound of formula (VIII) to obtain a compound of formula (IX);

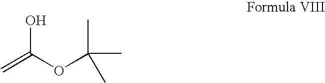

e). hydrolyzing a compound of formula (IX) to obtain a compound of formula (X);

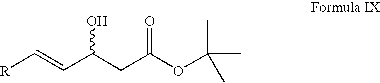

f). resolving a compound of formula (X), first converting the racemic compound to its diastereomeric salt using (+) or (−) enantiomeric amine of the formula (XI) and separating the mixture of diastereomeric salt into the individual diastereomers by chromatography or crystallization and then neutralizing the diastereomeric salt to obtain the enantiomerically pure products.

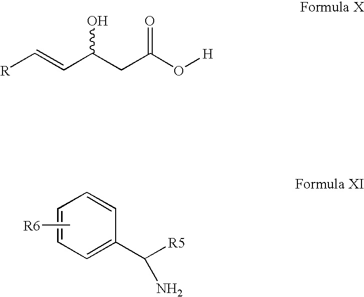

wherein, R5 represent C1-C4-alkyl, which is optionally substituted by hydroxyl; R6 represent hydrogen, halogen, C1-C4 alkyl or C1-C4 alkoxy;
g). esterifying a resulting compound of formula (XII) to obtain a compound of formula (XIII)

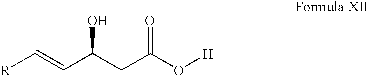

h). condensing a compound of formula (XIII)

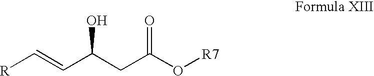

wherein, R7 is an aliphatic residue with a compound of formula (VIII) to obtain a compound of formula (XIV);

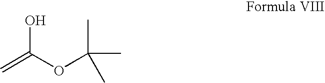

i). reducing a compound of formula (XIV) to obtain a compound of formula (XV)

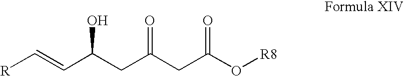

wherein, R8 represent C1-C4 alkyl;
j). hydrolysing a compound of formula (XV) and converting into a salt of formula I thereof

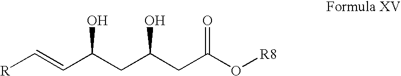

wherein R and R8 have the meanings as defined.

In reaction step (a), the reaction of a compound of formula II with a compound of formula III is carried out in a suitable inert solvent, preferably toluene at temperature range from 25° C. to reflux temperature of the solvent, preferably from 60° C. to reflux temperature of the solvent.

Reduction of formula V (step b) using diisobutylaluminium hydride (DIBAL) is carried out in a suitable inert solvent, especially toluene, and in a temperature range from −5° C. to +5° C., preferably at 0° C.

Oxidation of compound of formula VI (step c) is carried out in an inert solvent at −70° C. to 28° C., preferably between 0° C. to 28° C. using oxidizing agents like pyridium chlorochromate (PCC), pyridinium dichromate (PDC) and Swern oxidation method, preferably pyridinium dichromate.

Step (d) is carried out in the presence of a suitable base and in a suitable inert solvent, especially tetrahydrofuran, and in a temperature range from −78° C. to the reflux temperature of the solvent, preferably at room temperature.

A suitable base is selected from alkali metal hydride, alkane alkali metal in presence of diisopropylamine and alkali alkylsilazanes. Especially preferred is the use of n-butyl lithium in the presence of diisopropylamine.

The saponification (step e) is carried out by using a strong base, such as an alkali metal hydroxide, preferably NaOH or KOH, in aqueous aliphatic alcohol as solvent, preferably aqueous methanol, and in a temperature range from 25° C. to reflux temperature of solvent, preferably between 30° C. to 65° C. and acidifying the resulting reaction mixture.

Resolution of the racemate (step f) of compound of formula X in to optically pure antipodes is carried out by means of known methods for the separation of entiomers, for example by means of preparative chromatography using chiral supports (HPLC) or by crystallization using optically pure precipitating agents, for example (+) or (−) phenylalkylamine or substituted phenylalkylamine, preferably (R)-1-phenylethylamine in alcoholic solvents such as lower alkanol, preferably ethanol and recrystallising from a mixture of ketonic solvent and lower alkanol, preferably acetone and methanol at variable ratio followed by neutralization.

Esterification of compound of formula XII (step g) is carried out, in lower alcoholic solvent, especially C1-C3 alkanol, preferably methanol, in presence of acidic catalyst like inorganic acids or p-toluensulphonic acid or acidic resins, and in a temperature range from 0° C. to reflux temperature of solvent, preferably between 0° C. to 28° C.

Condensation step (step h) is carried out in the presence of a suitable base and in suitable inert solvent, especially tetrahydrofuran, and in a temperature range from −78° C., to the boiling point of the solvent, preferably at room temperature.

The suitable base is selected from alkane alkalimetal, like n-butyllithium in the presence of diisopropylamine, alkali alkylsilazanes. Especially preferred is the use of n-butyllithium in the presence of diisopropylamine.

The reduction of compound of formula XIV (step i), is carried out in a mixture of an inert solvent, preferably tetrahydrofuran and lower alkanol, preferably methanol, in the ratio of 4:1 volume/volume, and at −78° C. to 0° C., preferably −78° C. to −70° C. To split the corresponding boronic ester, the reaction mixture is then treated with methanol, at 0° C. to the boiling point of solvent, preferably in range of 0° C. to 40° C.

A preferred reduction agent is a hydride such as an alkali metal borohydride, especially sodium borohydride, in the presence of a di-C1-C7-alkyl-C1-C4 alkoxy-borane, preferably diethylmethoxyborane.

Isolation of compound of formula I (step j), is carried out first by saponification of compound of formula XV with a base, such as an alkali metal hydroxide, preferably NaOH followed by treatment with aqueous calcium chloride solution. The novel intermediates in the present invention are:

1) Compound of formula V

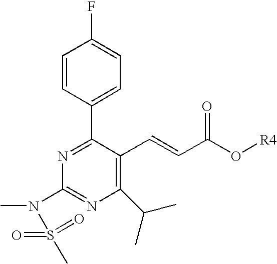

2) Compound of formula VI

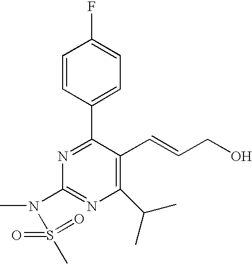

3. Compound of formula VII

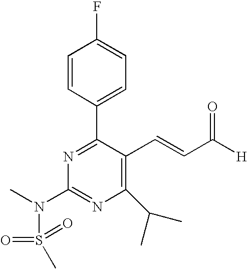

4. Compound of formula X

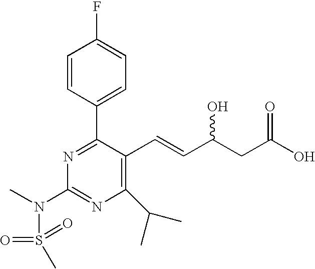

5. Compound of formula XII

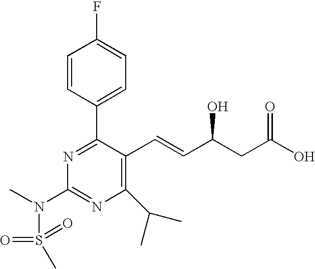

The starting material of formula (III) may be prepared, for example, as described in Bioorganic & Medicinal Chemistry 1997, 437.

In the following examples, the preferred embodiments of the present invention are described only by way of illustrating the process of the invention. However, these are not intended to limit the scope of the present invention in any way.

### Example 1

### Preparation of ethyl (2E)-3 {4-(4-flurophenyl)-6-isopropyl-2-[methyl (methylsulfonyl)amino]pyrimidin-5-yl } acrylate

To a solution of N-[4-(4-flurophenyl)-5-formyl-6-isopropylpyrimidin-2-yl]-N-methylmethylsulfonamide (55 g; 156 mmol) in 700 ml of toluene, 60.2 g of (carbethoxymethylene)triphenylphosphorane (172 mmol) was added at 25-29° C. The reaction mixture was refluxed for 6 hours. After completion of reaction (TLC; disappearance of starting material), reaction mixture was cooled between 25-28° C. and 500 ml of n-hexane was added and stirrer for 15 minutes. The separated solid was removed by filtration and the filtrate was distilled under reduced pressure to remove the solvents. The oily mass obtained after removal of solvents was purified through silica gel column chromatography to obtain ethyl (2E)-3-{4-(4-flurophenyl)-6-isopropyl-2-[methyl(methylsulfonyl)amino]pyrimidin-5-yl}acrylate as a solid.

¹H NMR (400 MHz. CDCl₃): 1.27-1.3 (9H, m, —CH(CH₃)₂, —CH₂CH₃), 3.33-3.4 (1H, m, —CH(CH₃)₂, 3.49 (3H, s, —NCH₃), 3.55 (3H, S, —SO₂CH₃), 4.19 (2H, q, —OCH₂CH₃), 5.81 (1H, d, J=16.10, C=CHCOOCH₂), 7.10 (2H, t, Ar—H), 7.59 (2H, dd, Ar—H), 7.68 (1H, d, J=16.10,—CH═CHCOOCH₂).

³C NMR (400 MHz, CDCl₃): 14.32, 21.97, 30.01, 32.29, 42.44, 60.76, 115.45, 115.67, 118.81, 125.71, 132.04, 132.73, 133.67, 133.71, 139.17, 157.97, 162.51, 164.33, 165.01, 165.50, 175.15

### Example 2

### Preparation of (2E)-3-{4-(4-flurophenyl)-6-isopropyl-2-[methyl(methyl sulfonyl)amino]pyrimidin-5-yl}propenol

A solution of ethyl (2E)-3-{4-(4-flurophenyl)-6-isopropyl-2-[methyl(methylsulfonyl) amino]pyrimidin-5-yl}acrylate (37 g; 87.8 mmol) in toluene (185 ml) was cooled to around −5° C. and with stirring. To this solution, DIBAL (20% in toluene; 159.3 ml; 193.3 mmol)) was added in drop wise over a period of approximately 2 hours under nitrogen atmosphere at temperature between −5° C. to +5° C. After stirred at this temperature for further 1 hour, to the reaction mixture 50 ml of acetic acid was added drop wise followed by 200 ml of water and 300 ml of ethyl acetate. The organic layer was separated and the aqueous layer was re extracted using 300 ml of ethyl acetate. The combined organic layers were washed twice with 500 ml of sat. NaHCO₃, twice with 500 ml of sat NaCl, dried over anhydrous Na₂SO₄, filtered and concentrated under reduced pressure to obtain (2E)-3-{4-(4-flurophenyl)-6-isopropyl-2-[methyl(methylsulfonyl)amino]pyrimidin-5-yl }propenol as a solid after complete removal of solvents.

¹H NMR (400 MHz. CDCl₃): 1.24 (6H, d, —CH(CH₃)₂, 1.69 (1H, br, s, —OH), 3.33-3.4 (1H, m, —CH(CH₃)₂, 3.49 (3H, s, —NCH₃), 3.54 (3H, s, —SO₂CH₃), 4.17 (2H, d, —CH₂OH), 5.63 (1H, dt, J=16.10, 5.0, ═CHCH₂OH), 6.56 (1H, d, J=16.10,—CH═CHCH₂OH), 7.06 (2H, t, Ar—H), 7.63 (2H, dd, Ar—H).

³C NMR (400 MHz, CDCl₃): 21.64, 31.93, 33.05, 42.34, 63.05, 114.09, 115.12, 121.20, 121.21, 123.60, 131.97, 132.05, 134.37, 134.35, 136.38, 157.20, 161.95, 163.46, 164.43, 174.84

### Example 3

### Preparation of (2E)-3-{4-(4-flurophenyl)-6-isopropyl-2-[methyl(methylsulfonyl)amino] pyrimidin-5-yl }propenal

A stirred slurry of chromium trioxide (49.15 g; 492 mmol) in 200 ml of dichloromethane was cooled to approximately 0° C. and pyridine (77.74 g) was added in dropwise manner over a period of 45 minutes at temperature between −5° C. to +5° C. After stirring for another 10 minutes, a solution of (2E)-3-{4-(4-flurophenyl)-6-isopropyl-2-[methyl(methylsulfonyl)amino]pyrimidin-5-yl}propenol (31 g; 82 mmol) in 200 ml of dichloromethane was added dropwise over a period of 45 minutes at 0 C. After completion of addition, the reaction mixture was stirrer for 2-3 hours at 0° C. Silica gel (100 g) was added and stirrer for 15 minutes. The reaction mixture was filtered and the solid was washed thrice with 200 ml of dichloromethane. The combined organic layers were washed with twice with 300 ml of 2.5% aqueous sodium hydroxide solution, 2.5% hydrochloric acid followed by saturated sodium chloride solution and dried over Na₂SO₄ The filtrate obtained after filtration was distilled under vacuum to get (2E)-3-{4-(4-flurophenyl)-6-isopropyl-2-[methyl(methylsulfonyl)amino]pyrimidin-5-yl}propenal as a yellow coloured solid.

¹H NMR (400 MHz. CDCl₃): 1.29 (6H, d, —CH(CH₃)₂, 3.33-3.4 (1H, m, —CH(CH₃)₂), 3.5 (3H, s, —NCH₃), 3.57 (3H, s, —SO₂CH₃), 6.18 (1H, dd, J=16.22, ═CHCHO), 7.12 (2H, t, Ar—H), 7.52 (1H, d, J=16.10,—CH═CHCHO), 7.57 (2H, dd, Ar—H), 9.58 (1H, d, —CHO). ³C NMR (400 MHz, CDCl₃): 21.25, 32.20, 33.06, 42.44, 115.58, 115.79, 131.83, 131.98, 133.44, 133.48, 135.24, 135.95, 147.10, 149.72, 158.22, 162.57, 164.78, 165.07, 175.18, 192.70.

### Example 4

### Preparation of racemic tert-butyl (4E)-5-{4-(4-flurophenyl)-6-isopropyl-2-[methyl(methylsulfonyl)amino]pyrimidin-5-yl}3hydroxy-4-pentenoate

Diisopropylamine (13.55 g (134 mmol) was taken in 100 ml of dry THF and cooled to —5 to 0° C. with stirring under nitrogen atmosphere. To this stirred solution n-butyllithium (1.6M in hexane; 86 ml; 134 mmol)) was added in drop wise manner over a period of approximately 30 minutes at temperature between −5° C. to +5° C. under nitrogen atmosphere. The reaction mixture was then allowed to reach +10° C. (in the course of 10 minutes) and maintained at that temperature for 30 min. Again the reaction mixture was cooled to around −65° C., tert-butyl acetate (15.56 g; 134 mmol) was added dropwise over a period of 20 minutes and stirred out at that temperature for 40 minutes. To this a solution of (2E)-3-{4-(4-flurophenyl)-6-isopropyl-2-[methyl(methylsulfonyl)amino]pyrimidin-5-yl}-propenal (23 g; 61 mmol) in 100 ml of dry THF was added dropwise over a period of 30 minutes. The reaction mixture was stirred out at temperature between −60 and −65° C., the reaction mixture was allowed to warm up to −5° C. (in time interval of −45 minutes) and stirred at that temperature for further 30 minutes. The reaction mixture was quenched with drop wise addition of acetic acid (50 ml) and stirred for ˜10 minutes. To this 200 ml of ethyl acetate was added followed by 200 ml of water and stirring is carried out for another 10 minutes. The layers were separated and the organic layer was discarded. The aqueous phase was extracted twice with 200 ml of ethyl acetate and the combined organic layers were washed twice with 300 ml of 5% aqueous NaHCO₃ solution and then with ˜5% sodium chloride solution, dried over anhydrous Na₂SO₄ and filtered. The filtrate was distilled under reduced pressure to obtain racemic tert-butyl (4E)-5-{4-(4-flurophenyl)-6-isopropyl-2-[methyl(methylsulfonyl)amino]pyrimidin-5-yl}-3-hydroxy-4-pentenoate as a pale brown oily mass.

¹H NMR (400 MHz. CDCl₃): 1.23 (6H, d, —CH(CH₃)₂, 1.42 (9H, s, —O—C(CH₃)₃), 2.22-2.42 (2H, m, —CH₂COO-), 3.3-3.36 (1H, m, —CH(CH₃)₂), 3.49 (3H, s, —NCH₃), 3.54 (3H, s, —SO₂CH₃), 4.48-4.52 (1H, m, —CHOH), 5.46 (1H, dd, J=16.10, 5.12, ═CHCHOH), 6.62 (1H, d, J=16.10,—CH═CHCHOH), 7.06 (2H, t, Ar—H), 7.62 (2H, dd, Ar—H).

### Example 5

### Preparation of racemic (4E)-5-{4-(4-flurophenyl)-6-isopropyl-2-[methyl(methylsulfonyl)amino]pyrimidin-5-yl}-3-hydroxy-4-pentenoic acid

To a stirred solution of tert-butyl (4E)-5-{4-(4-flurophenyl)-6-isopropyl-2-[methyl(methylsulfonyl)amino]pyrimidin-5-yl}-3hydroxy-4-pentenoate (28.5 g; 57 mmol) in 200 ml of methanol, a solution of aqueous sodium hydroxide (2.54 g; 63.5 mmol in 50 ml of water) was added slowly at temperature between 27-29° C. The reaction mixture was heated and refluxed for 6-10 hours. After completion of reaction (completion of reaction was monitored by TLC, ethyl acetate: hexane 3:7), 50 ml of water and 200 ml of tert-butyl methyl ether were added. The organic layer was separated and washed with 100 ml water. The aqueous layers were combined and the pH was adjusted to approximately between 3-4 by acidification and extracted twice with 200 ml of dichloromethane. The combined organic layers were washed with 100 ml saturated NaCl solution, dried over anhydrous Na₂SO₄ The filtrate obtained after filtration was evaporated to dryness under vacuum to obtain racemic (4E)-5-{4-(4-flurophenyl)-6-isopropyl-2-[methyl(methylsulfonyl)amino] pyrimidin -5-yl}-3-hydroxy-4-pentenoic acid as a white solid.

¹H NMR (400 MHz, CDCl₃): 1.2 (6H, d, —CH(CH₃)₃), 2.45-2.52 (1H, m, —CH₂-COOH), 3.27-3.33 (1H, m, —CH(CH₃)₃), 3.49 (3H, s, —NCH₃), 3:54 (3H, s, —SO₂CH₃), 4.58 (1H, s, >CH-OH), 5.46 (1H, dd, J=15.98, ═CHCOOH), 6.7 (1H, d, J=15.85,—CH═CHCOOH), 7.1 (2H, t, Ar—H), 7.59 (2H, dd, Ar—H).

### Example 6

### Preparation of (4E)-5-{4-(4-flurophenyl)-6-isopropyl-2-[methyl (methylsulfonyl)amino]pyrimidin-5-yl} (3S)-3-hydroxy-4-pentenoic acid

To a solution of racemic (4E)-5-{4-(4-flurophenyl)-6-isopropyl-2-[methyl(methylsulfonyl)amino]pyrimidin-5-yl}-3-hydroxy-4-pentenoic acid in ethanol, (R)-1-phenyl ethylamine was added at 25-29° C. The reaction mixture was cooled to 0° C. and stirred for another 3 hours. The solid precipitated was filtered and washed with tert-butyl methyl ether, dried under vacuum. The solid obtained after drying was recrystallised from 5 volumes of methanol-acetone mixture (1:4 ratio by v/v) to get (R)-1-phenylethylamine salt of (4E)-5-{4-(4-flurophenyl)-6-isopropyl-2-[methyl(methylsulfonyl)amino] pyrimidin-5-yl}(3 S)-3-hydroxy-4-pentenoic acid.

The crystallized salt was taken in methanol and treated with aqueous sodium hydroxide solution at 25-28° C. with stirring. After stirring for 1 hour, water was added followed by tert-butyl methyl ether. The organic layer was separated and the aqueous layer was acidified (pH of 3-4) and extracted with dichloromethane. After removal of solvent under vacuum, (4E)-5-{4-(4-flurophenyl)-6-isopropyl-2-[methyl(methylsulfonyl)amino] pyrimidin-5-yl} (3S)-3-hydroxy-4-pentenoic acid was obtained as a solid.

¹H NMR (400 MHz, CDCl₃): 1.2 (6H, d, —CH(CH₃)₃), 2.45-2.52 (1H, m, —CH₂—COOH), 3.27-3.33 (1H, m, —CH(CH₃)₃), 3.49 (3H, s, —NCH₃), 3.54 (3H, s, —SO₂CH₃), 4.58 (1H, s, >CH—OH), 5.46 (1H, dd, J=15.98, ═CHCOOH), 6.7 (1H, d, J=15.85,—CH═CHCOOH), 7.1 (2H, t, Ar—H), 7.59 (2H, dd, Ar—H).

¹³C NMR (400 MHz, CDCl₃): 21.55, 32.11, 33.10, 40.40, 42.37, 68.09, 114.96, 115.16, 120.86, 124.22, 131.99, 132.08, 134.27, 134.30, 137.32,157.34, 161.99, 163.53, 164.47, 174.82, 176.81.

### Example 7

### preparation of methyl (4E)-5-{4-(4-flurophenyl)-6-isopropyl-2-[methyl(methylsulfonyl) amino]pyrimidin-5-yl}(3S)-3-hydroxy-4-pentenoate

Methanol (25 ml) was taken in a 100 ml three necked round bottomed flask and cooled to −5° C. with stirring. To this acetyl chloride (0.588 g; 7.488 mmol) was added dropwise in such a way that the temperature remains between −5° C. to +5° C. over a period of approximately 10 minutes. After stirred for 30 minutes at 0° C., a solution of (4E)-5-{4-(4-flurophenyl)-6-isopropyl-2-[methyl(methylsulfonyl)amino]pyrimidin-5-yl}(3S)-3-hydroxy-4-pentenoic acid (4.2 g; 9.6 mmol) in 15 ml of methanol was added dropwise over a period of 10 minutes at 0° C. and is maintain that temperature for further 30 minutes. Then the reaction mixture was allowed to warm at 20-25° C. and stirred for 3-4 hours at 25-29° C. Again the reaction mixture was cooled to 0° C. and 3 g of powered NaHCO₃ was added in portions. The reaction mixture was filtered and to the filtrate 50 ml of ethyl acetate and 30 ml of water were added. The layers were separated and the aqueous layer was extracted twice with 30 ml of ethyl acetate. The combined organic layers were washed with 50 ml of saturated NaHCO₃ solution, 50 ml of saturated NaCl solution and dried over anhydrous Na₂SO₄ Methyl (4E)-5-{4-(4-flurophenyl)-6-isopropyl-2-[methyl (methylsulfonyl)amino]pyrimidin-5-yl}(3S)-3-hydroxy-4-pentenoate was obtained as solid after complete removal of solvent by distillation under vacuum.

¹H NMR (400 MHz, CDCl₃): 1.2 (6H, d, —CH(CH₃)₃), 2.4-2.5 (2H, m, —CH₂COOMe), 3.1 (1H, d, >CH—OH), 3.34-3.41 (1H, m, —CH(CH₃)₃), 3.48 (3H, s, -NCH₃), 3.54 (3H, s, -SO₂CH₃), 3.7 (3H, s, —COOCH₃), 4.6 (1H, s, >CH—OH), 5.5 (1H, dd, J=16.10, 5.12 ═CHCOOCH₃), 6.6 (1H, d, J=16.10,—CH═CHCOOMe), 7.1 (2H, t, Ar—H), 7.6 (2H, dd, Ar—H).

³C NMR (400 MHz, CDCl₃): 21.54, 32.03, 33.04, 40.31, 51.85, 68.15, 114.89, 115.10, 121.00, 123.73, 132.00, 132.09, 134.32, 137.71, 157.27, 161.94, 164.42, 172.38, 174.79.

### Example 8

### Preparation of tert-butyl (6E)-7-{4-(4-flurophenyl)-6-isopropyl-2-[methyl(methylsulfonyl) amino]pyrimidin-5-yl }(5S)-5-hydroxy-3-oxo-6-heptenoate

To a solution of diisopropylamine (0.9 g; 8.87 mmol) in 10 ml of dry tetrahydrofuran, n-butyllithium (1.6M in hexane; 6 ml; 8.87 mmol) was added at 0° C. under nitrogen atmosphere, with stirring in dropwise over a period of ˜10 minutes. The reaction mixture was then allowed to warm up to +10° C. and maintained at that temperature for 30 minutes. Again the reaction mixture was cooled to −65° C. and tert-butyl acetate (1.03 g; 8.87 mmol) was added dropwise over a period of 5 minutes. After stirred for another 40 minutes, the resulting solution was transferred to a solution of methyl (4E)-5-{4-(4-flurophenyl)-6-isopropyl-2-[methyl(methylsulfonyl)amino]pyrimidin-5-yl}(3S)-3-hydroxy-4-pentenoate (1 g; 2.2 mmol) in 5 ml of dry THF at 0° C.

The reaction mixture was allowed to reach to 20° C. and stirred at that temperature for ˜4 hours. 1 ml of acetic acid was added in dropwise to the reaction mixture followed by 10 ml of ethyl acetate and 10 ml of water. After stirring for ˜10 minutes, the layers were separated and the aqueous phase was extracted twice with 30 ml of ethyl acetate. The combined organic layers were washed twice with 30 ml of saturated NaHCO₃ solution and then with saturated NaCl solution, dried over anhydrous Na₂SO₄ The filtrate obtained after filtration was distilled under vacuum to remove the solvent completely. tert-butyl (6E)-7-{4-(4-flurophenyl)-6-isopropyl-2-[methyl(methylsulfonyl)amino]pyrimidin-5-yl}-(5R)-5-hydroxy-3-oxo-6-heptenoate was obtained as an orange oily mass and was taken as it is for next step.

### Example 9

Preparation of tert-butyl (6E)-7-{4-(4-flurophenyl)-6-isopropyl-2-[methyl(methyl sulfonyl)amino]pyrimidin-5-yl}(3R,5S)-3,5-dihydroxyhept-6-enoate tert-Butyl (6E)-7-{4-(4-flurophenyl)-6-isopropyl-2-[methyl(methylsulfonyl)amino] pyrimidin-5-yl}(5S)-5-hydroxy-3-oxo-6-heptenoate (1 g; 1.87 mmol) was taken in 10 ml of dry THF/methanol (4:1) and cooled to −78° C. under nitrogen atmosphere with stirring. To this stirred solution of diethylmethoxyborane (1 M in THF; 2.1 g; 2.05 mmol)) was added dropwise over a period of ˜5 minutes. After stirring for at that temperature for further 30 minutes, NaBH₄ (0.08 g; 2.05 mmol) was added at −78° C. The reaction mixture was stirred at −78° C. for 3-4 hours. To the reaction mixture 1 ml of acetic acid was added in dropwise followed by 10 ml of ethyl acetate and 10 ml of water. After stirring for 10 minutes at −78° C. the reaction mixture was allowed reach 25-28° C. The layers were separated and the aqueous layer was extracted twice with 30ml of ethyl acetate. The combined organic phases were washed twice with 30 ml saturated NaHCO₃ solution and then with saturated NaCl solution, dried over anhydrous Na₂SO₄ The reaction mixture was filtered and the solvent was removed by distillation under vacuum. The oily product thus obtained was swapped thrice with 30 ml of methanol to remove borate complex and concentrated to obtain an oily mass, which after column purification provided tert-butyl (6E)-7-{4-(4-flurophenyl)-6-isopropyl-2-[methyl(methylsulfonyl)amino]pyrimidin-5-yl}(3R,5S)-3,5-dihydroxyhept-6-enoate as a solid.

¹H NMR (400 MHz, CDCl₃): 1.23 (6H, d, —CH(CH₃)₃), 1.4-1.5 (11H, —CH₂—C(CH₃)₃), 2.34 (2H, d, —CH₂COO), 3.31-3.38 (1H, m, —CH(CH₃)₃), 3.49 (3H, s, —NCH₃), 3.54 (3H, s, —SO₂CH₃), 3.76 (H, bs, —OH), 3.86 (H, bs, —OH), 4.14 (1H, d, >CH—OH), 4.42 (1H, t, >CH—OH), 5.42 (1H, dd, J=15.98 ═CHCOO), 6.6 (1H, d, J=16.10,—CH═CHCOO), 7.06 (2H, t, Ar—H), 7.63 (2H, dd, Ar—H).

### Example 10

### Preparation of calcium (2:1)-(+)7-{4-(4-flurophenyl)-6-isopropyl-2-[methyl(methylsulfonyl)amino]pyrimidin-5-yl}(3R,5S)-3,5-dihydroxy-(E)-hept-6-enoic acid

A solution of tert-butyl (6E)-7-{4-(4-flurophenyl)-6-isopropyl-2-[methyl(methyl sulfonyl)amino]pyrimidin-5-yl}(3R,5S)-3,5-dihydroxyhept-6-enoate (2 g; 3.72 mmol) in 30 ml of acetonitrile of 0.25 M solution of NaOH (14.9 ml; 3.72 mmol) was added over a period of 5 minutes at temperature between 26-29° C. with stirring. After stirred for 3-4 hours, 30 ml of tert-butyl methyl ether was added followed by 10 ml of water. The layers were separated and the organic layer was extracted with 20 ml of water. The combined aqueous layers were concentrated by evaporation under reduced pressure to its half volume. To the concentrated aqueous layer, 1 M solution of CaCl₂.2H₂O (1.86ml; 1.86 mmol) was added dropwise with stirring at 25-28° C. After stirred for 45 minutes, the precipitate formed was filtered and washed with water to obtain Rosuvastatin calcium as a white solid.

1. A process for the manufacture of Rosuvastatin of formula I, according to the present invention, comprising 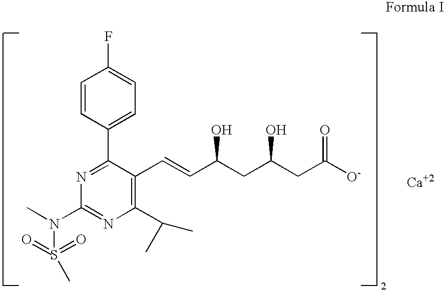
a) reacting a compound of formula (II) 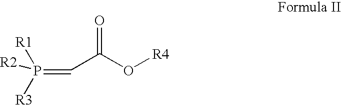
wherein, R1, R2, R3, are substituted or unsubstituted phenyl and R4 is an aliphatic residue selected from C1-C4 alkyl,
with a compound of formula R—CHO (III) wherein R represents the following structure (Formula IV) to obtain a compound of formula V; 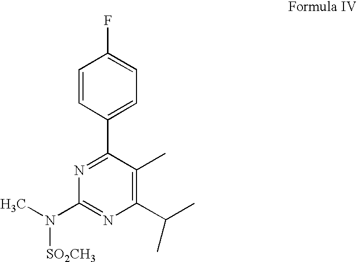
b). reducing a compound of formula (V) to obtain a compound of formula (VI); 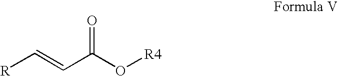
c). oxidising a compound of formula (VI) to obtain a compound of formula (VII) 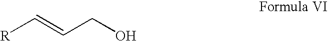
d). adding a compound of formula (VII) 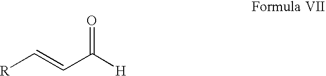
with a compound of formula (VIII) to obtain a compound of formula (IX); 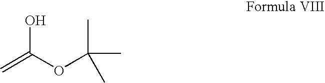
e). hydrolyzing a compound of formula (IX) to obtain a compound of formula (X); 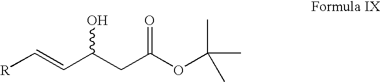
f). resolving a compound of formula (X), first converting the racemic compound to its diastereomeric salt using (+) or (−) enantiomeric amine of the formula (XI) and separating the mixture of diastereomeric salt into the individual diastereomers by chromatography or crystallization and then neutralizing the diastereomeric salt to obtain the enantiomerically pure products. 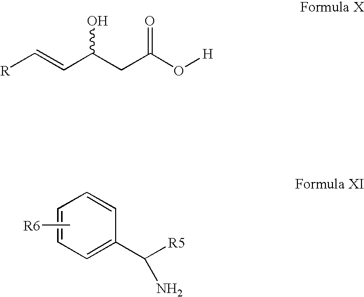
wherein, R5 represent C1-C4-alkyl, which is optionally substituted by hydroxyl; R6 represent hydrogen, halogen, C1-C4 alkyl or C1-C4 alkoxy;
g). esterifying a resulting compound of formula (XII) to obtain a compound of formula (XIII) 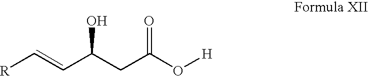
h). condensing a compound of formula (XIII) 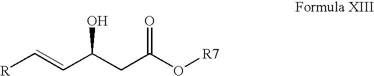
wherein, R7 is an aliphatic residue
with a compound of formula (VIII) to obtain a compound of formula (XIV); 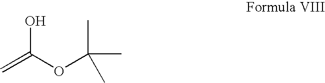
i). reducing a compound of formula (XIV) to obtain a compound of formula (XV) 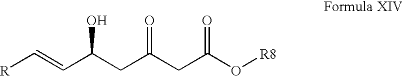
wherein, R8 represent C1-C4 alkyl;
j). hydrolysing a compound of formula (XV) and converting into a salt of formula I thereof 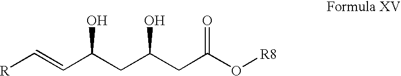
wherein R and R8 have the meanings as defined.

2. A process according to claim 1, wherein the compound of formula II, V, XIII, wherein R4 or R7, respectively, represent C1-C4 alkyl and preferably methyl or ethyl. 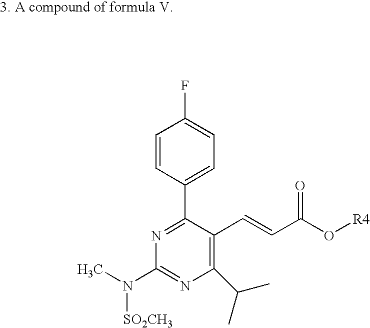
wherein R4 is defined in claim 1

4. A compound of formula VI.

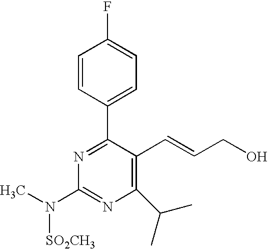

5. A compound of formula VII.

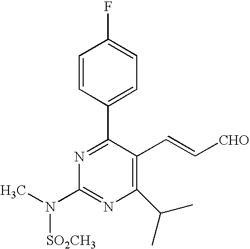

6. A compound of formula X.

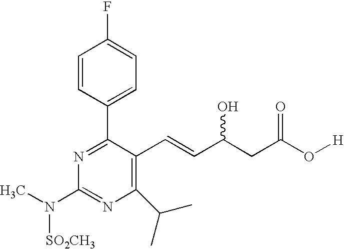

7. A compound of formula XII

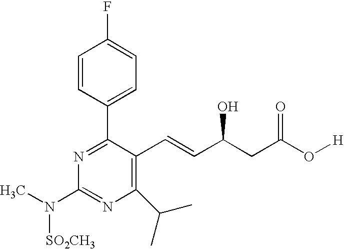

8. A process according to claim 1, the reaction of a compound of formula II with a compound of formula III is carried out in a suitable inert solvent, preferably toluene at temperature range from 25° C. to reflux temperature of the solvent, preferably from 60° C. to reflux temperature of the solvent.

9. A process according to claim 1, reduction of formula V (step b) using diisobutylaluminium hydride (DIBAL) is carried out in a suitable inert solvent, especially toluene, and in a temperature range from −5° C. to +5° C., preferably at 0° C.

10. A process according to claim 1, oxidation of compound of formula VI (step c) is carried out in an inert solvent, preferably dichloromethane at −70° C. to 28° C., preferably between 0° C. to 28° C. using oxidizing agents like pyridinium cholrochromate, pyridinium dichromate and Swern oxidation method, preferably pyridinium dichromate.

11. A process according to claim 1, Step (d) is carried out in the presence of a suitable base and in a suitable inert solvent, especially tetrahydrofuran, and in a temperature range from −78° C. to the reflux temperature of the solvent, preferably at room temperature. A suitable base is selected from alkali metal hydride, alkali alkylsilazanes, alkane alkali metal in presence of diisopropylamine, especially preferred is the use of n-butyllithium in the presence of diisopropylamine.

12. A process according to claim 1, the saponification step e) is carried out by using a strong base, such as an alkali metal hydroxide, preferably NaOH or KOH, in aqueous aliphatic alcohol as solvent, preferably aqueous methanol, and in a temperature range from 25° C. to reflux temperature of solvent, preferably between 30° C. to 65° C. and acidifying the resulting reaction mixture.

13. A process according to claim 1, resolution of the racemate (step f) of compound of formula X in to optically pure antipodes is carried out by means of known methods for the separation of entiomers, for example by means of preparative chromatography using chiral supports (HPLC) or by crystallization out using optically pure precipitating agents, for example (+) or (−) phenylalkylamine or substituted phenylalkylamine, preferably (R)-1-phenylethylamine in alcoholic solvents such as lower alkanol, preferably ethanol and recrystallising from a mixture of ketonic solvent and lower alkanol, preferably acetone and methanol at variable ratio followed by neutralization.

14. A process according to claim 1, esterification of compound of formula XII (step g) is carried out, in lower alcoholic solvent, especially C1-C3 alkanol, preferably methanol, in presence of acidic catalyst like inorganic or p-toluensulphonic acid or acidic resins, and in a temperature range from 0° C. to reflux temperature of solvent, preferably between 0° C. to 28° C.

15. A process according to claim 1, condensation step (step h) is carried out in the presence of a suitable base and in a suitable inert solvent, especially tetrahydrofuran, and in a temperature range from −78° C., to the boiling point of the solvent, preferably at room temperature. The suitable base is selected from alkane alkalimetal, like n-butyllithium in the presence of diisopropylamine, alkali alkylsilazanes. Especially preferred is the use of n-butyl-lithium in the presence of diisopropylamine.

16. A process according to claim 1, the reduction of compound of formula XIV (step i), is carried out in a mixture of an inert solvent, preferably tetrahydrofuran and lower alkanol, preferably methanol, in the ratio of 4:1 volume/volume, and at −78° C. to 0° C., preferably −78° C. to −70° C. To split the corresponding boronic ester, the reaction mixture is then treated with methanol, at 0° C. to the boiling point of solvent, preferably in range of 0° C. to 40° C. A preferred reduction agent is a hydride such as an alkali metal borohydride, especially sodium borohydride, in the presence of a di-C1-C7-alkyl-C1-C4 alkoxyborane, preferably diethylmethoxyborane.

17. A process according to claim 1, isolation of compound of formula I (step j), is carried out first by saponification of compound of formula XV with a base, such as an alkali metal hydroxide, preferably NaOH followed by treatment with aqueous calcium chloride solution.
